(19) United States
(12) Patent Application Publication
(10) Pub. No.: US 2015/0238410 A1
(43) Pub. Date: Aug. 27, 2015

(54) ULTRASOUND ANTI STRETCH MARK GEL COMPOSITION

(71) Applicant: Mila Klevtsov, Wayne, NJ (US)

(72) Inventor: Mila Klevtsov, Wayne, NJ (US)

(21) Appl. No.: 14/191,741

(22) Filed: Feb. 27, 2014

(51) Int. Cl. A61K 8/97; A61K 8/81; A61K 8/34; A61Q 19/00; B65D 47/20; A61K 49/22

(52) U.S. Cl. CPC A61K 8/97; B65D 47/2031; A61K 49/22; A61K 8/345; A61K 8/347; A61Q 19/00; A61K 8/8147; A61K 2800/5922

(57) ABSTRACT

An improved ultrasound imaging gel for topical use and a method for create such comprising: water, propylene glycol, glycerin, phenoxyethanol or Carbopol® 940 Polymer, perfumes and dyes combined with equisetum arvense, alchemilla vulgaris, and hedera helix. The improved ultrasound imaging gel may be dispensed from a bottle, where the bottle includes: a body portion, where the body portion includes a slight indenture at a top diameter; a nipple, where the nipple includes an opening for the dispensing of the gel through the opening; and a ribbed base surrounding the nipple.

### BACKGROUND OF THE INVENTION

1. Field of Invention

The present invention relates to a gel composition that is used for ultrasound applications where the gel includes anti-stretch mark properties.

2. Description of Related Art

An ultrasound examination involves is a process of reviewing soft tissue through ultrasound medical imaging also known as diagnostic sonography. This diagnostic imaging that is created with the ultrasound system provides a means for medical imaging to examine the internal organs of humans or animals. One common application of ultrasound imaging relates to use during pregnancy to view an image of a fetus. The ultrasound imaging sonograph that is used includes a probe with tranducers that is applied to the skin of the individual that uses the ultrasound sonographic waves to create the image on a video display. Prior to the placement of the ultrasound device onto the skin of the patient the technician or doctor normally applies a gel coating to help in the transmission and detection of the images within the organs. This gel coating is commonly used optimizes the operation of the sonograph. The ultrasound imaging is used throughout the pregnancy of a woman and therefore it is a procedure that a woman must endure several times during her pregnancy. The gel that is typically used has no further benefit for the patient. One aesthetic drawback to pregnancies is the numerous stretch marks that result after and during the pregnancy on the mother's skin. Most women address these stretch marks by using topical creams and gels are used to reduce the stretch marks visual effects. It would therefore be advantageous to have an ultrasound gel that includes stretch mark prevention attributes. This type of improvement allows the user to gain some further benefit of the application of the ultrasound gel over the skin during a ultrasound sonographic examination.

### SUMMARY OF THE INVENTION

An improved ultrasound imaging gel for topical use and a method for create such comprising: water, propylene glycol, glycerin, phenoxyethanol or Carbopol® 940 Polymer, perfumes and dyes combined with equisetum arvense, alchemilla vulgaris, and hedera helix. The improved ultrasound imaging gel may be dispensed from a bottle, where the bottle includes: a body portion, where the body portion includes a slight indenture at a top diameter; a nipple, where the nipple includes an opening for the dispensing of the gel through the opening; and a ribbed base surrounding the nipple.

### BRIEF DESCRIPTION OF DRAWINGS

FIG. 1 depicts a dispensing bottle for an ultrasound anti stretch mark gel in accordance with the present invention.

Further aspects and features of the present disclosure will become apparent from the detailed description provided hereinafter. In addition, any one or more aspects of the present disclosure may be implemented individually or in any combination with any one or more of the other aspects of the present disclosure. It should be understood that the detailed description and specific examples, while indicating exemplary embodiments of the present disclosure, are intended for purposes of illustration only and are not intended to limit the scope of the present disclosure.

### DETAILED DESCRIPTION

The present invention relates to an improved ultrasound gel that includes anti stretch mark attributes. The common or conventional ultrasound gel includes a basic chemical composition that includes the following ingredients: water, propylene glycol, glycerin, phenoxyethanol or carbapol R940 polymer, perfumes and dyes. This particular composition is commonly used in the ultrasound gel as a means to effectively use the sonograph during use. By adding an anti-stretch gel composition this common composition for ultrasound gel may be improved upon. The improving aspects of ingredients of the present invention include equisetum arvense (commonly known as horse tail) used for skin softening; alchemilla vulgaris for healing properties; and hedera helix (ivy) for improved skin elasticity. The combination of these three critical ingredients into the common ultrasound gel creates an anti stretch mark characteristic incorporated into the ultrasound gel.

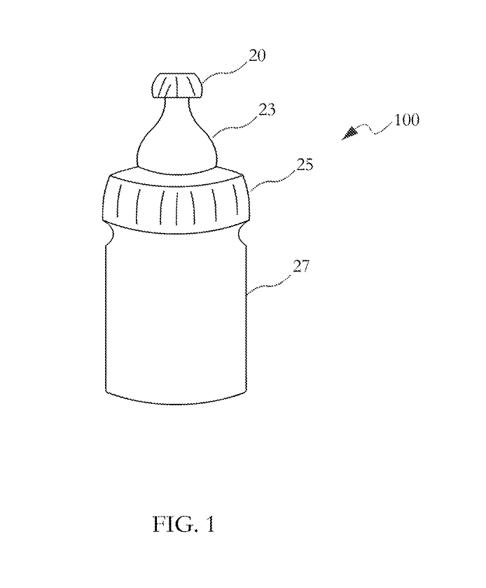

Further in reference to FIG. 1 a portable plastic bottle 100 may be utilized to dispense the ultrasound anti stretch gel in accordance with the present invention. The bottle 100 includes a body portion 27, which has a slight indenture at the top diameter. A nipple 23 is provided with an opening 20 for the dispensing of the gel through the opening 20. The nipple includes a ribbed base 25. Preferably the ultrasound anti stretch gel in accordance with the present invention may be placed in this bottle 100 and then used over the skin of a patient during an ultrasound imaging examination. The present invention includes both the composition as described above and a method of creating an ultrasound gel with anti-stretch properties. The instant invention has been shown and described in what it considers to be the most practical and preferred embodiments. It is recognized, however, that departures may be made there from within the scope of the invention and that obvious modifications will occur to a person skilled in the art.

What is claimed is:

1. An improved ultrasound imaging gel for topical use comprising: water, propylene glycol, glycerin, phenoxyethanol, perfumes and dyes combined with equisetum arvense, alchemilla vulgaris, and hedera helix.

2. The improved ultrasound imaging gel according to claim 1, further comprising a bottle for dispensing the gel where the bottle includes: a body portion 27, where the body portion includes a slight indenture at a top diameter; a nipple, where the nipple includes an opening for the dispensing of the gel through the opening; and a ribbed base surrounding the nipple.

3. An improved ultrasound imaging gel for topical use comprising: water, propylene glycol, glycerin, Carbopol® 940 Polymer, perfumes and dyes combined with equisetum arvense, alchemilla vulgaris, and hedera helix.

4. The improved ultrasound imaging gel according to claim 3, further comprising a bottle for dispensing the gel where the bottle includes: a body portion 27, where the body portion includes a slight indenture at a top diameter; a nipple, where the nipple includes an opening for the dispensing of the gel through the opening; and a ribbed base surrounding the nipple.

5. A method of creating an anti-stretch mark ultrasound imaging gel comprising the steps of:
a. combining water, propylene glycol, glycerin, phenoxyethanol, perfumes and dyes; and
b. adding equisetum arvense, alchemilla vulgaris, and hedera helix, wherein the step of adding contributes anti-stretch mark attributes to the ultrasound imaging gel.

6. The method of creating an anti-stretch mark ultrasound imaging gel further includes substituting Carbopol® 940 Polymer for the phenoxyethanol in the step of combining
